(19) United States
(12) Patent Application Publication
(10) Pub. No.: US 2026/0250883 A1
(43) Pub. Date: Aug. 27, 2026

(54) LIPID NANOPARTICLE PRODUCTION SYSTEM AND METHOD OF MONITORING AND CONTROLLING THE SAME

(71) Applicant: Sartorius Stedim Biotech GmbH, Göttingen (DE)

(72) Inventors: David Pollard, Stow, MA (US); Michael Olszowy, Erie, CO (US); Mehdi Dehghani, Medford, MA (US); Samin Akbari, Winchester, MA (US); Fujun Wang, Waltham, MA (US); Yuji Takeda, Wellesley, MA (US)

(73) Assignee: Sartorius Stedim Biotech GmbH, Göttingen (DE)

(21) Appl. No.: 18/993,397

(22) PCT Filed: Jul. 11, 2023
(86) PCT No.: PCT/EP2023/069146

§ 371 Date: Jan. 10, 2025

Related U.S. Application Data
(60) Provisional application No. 63/388,358, filed on Jul. 12, 2022.
(60) Provisional application No. 63/390,799, filed on Jul. 20, 2022.

(51) Int. Cl. C40B 50/08; G16C 20/62; G16C 60/00

(52) U.S. Cl. CPC C40B 50/08; G16C 20/62; G16C 60/00

(57) ABSTRACT

The present invention relates to automated nanoparticle synthesis systems and computer-implemented methods of monitoring and controlling a process of manufacturing lipid nanoparticles (LNPs) containing nucleic acid cargo. The present invention furthermore relates to a computer program product comprising computer-readable instructions, which, when loaded and executed on a computer system, causes the computer system to perform operations according to said methods.

### FIELD OF THE INVENTION

The present invention relates to automated nanoparticle synthesis systems and computer-implemented methods of monitoring and controlling a process of manufacturing lipid nanoparticles (LNPs) containing nucleic acid cargo. The present invention further relates to a computer program product comprising computer-readable instructions, which, when loaded and executed on a computer system, causes the computer system to perform operations according to said methods.

### BACKGROUND OF THE INVENTION

In recent years, LNPs containing nucleic acid cargo have gained considerable attention, e.g., due to the development of messenger ribonucleic acid (mRNA)-based vaccines. Hereby, mRNA (or any other therapeutic nucleic acid of interest) is encapsulated in LNPs for a stable delivery to and efficient transfection of target cells. Generally, LNPs protect their cargo from degradation, deliver therapeutics to target cells or tissues, and control the release of the cargo at the desired location.

LNPs are typically composed of amino lipids (ionizable or cationic amino lipids) as the main component, as well as phosphatidylcholine lipids, cholesterol, and polyethylene glycol-lipid conjugates (PEG-lipids). In the manufacturing process, a solution containing nucleic acid cargo is mixed with a solution containing lipids at an acidic pH, followed by neutralization, buffer exchange, and concentration to obtain the LNPs containing the nucleic acid cargo.

However, so far, there are several challenges involved in the manufacturing development and screening of LNPs since methods for the high throughput production of LNPs that would allow for the fast and easy generation and subsequent screening and analysis of a variety of LNPs are lacking. In particular, larger scale screening of LNPs is hampered by current production methods which allow for the generation of roughly less than 20 different LNPs per person per day, with offline assays for the analysis of as less as three or four parameters of said LNPs taking two to three days to complete. An enhanced controlling during the manufacturing process would also improve the scaling up of said process from a laboratory scale (<2 mg) to industrial scale (>2 g).

Therefore, a strong need exists to provide a method for the high throughput preparation and analysis of LNPs containing a nucleic acid cargo. Also, a strong need exists to provide a method of monitoring and controlling a process of manufacturing lipid nanoparticles containing nucleic acid cargo in real-time for increasing robustness and reproducibility of the process.

This need is satisfied by providing the embodiments characterized in the claims.

### SUMMARY OF THE INVENTION

The present invention relates to the following items:
1. A computer-implemented method for the high throughput preparation of lipid nanoparticles (LNPs) containing a nucleic acid cargo, comprising the steps of
   (a) providing one or more solution(s) comprising a nucleic acid cargo;
   (b) providing one or more solution(s) comprising a lipid;
   (c) mixing the one or more solution(s) comprising a nucleic acid cargo with the one or more solution(s) comprising a lipid at an acidic pH;
   (d) neutralizing the pH of the mixed solution obtained in step (c) via dilution to stabilize the LNPs obtained in step (c);
   (e) performing a buffer exchange on the neutralized solution obtained in step (d) via ultrafiltration and/or diafiltration (UF/DF) to concentrate the LNPs obtained in step (d) and reformulate the same into a cryogenic buffer, thereby generating one or more LNP preparation(s);
   wherein
   during each of steps (c) to (e), as well as after step (e), one or more of the particle size, polydispersity, and nucleic acid encapsulation of the one or more LNPs are monitored in real-time, thereby generating a data set for each LNP preparation; and the generated data set is associated with the respective LNP preparation.
2. The computer-implemented method of item 1, wherein two or more individual LNP preparations are generated in parallel.
3. The computer-implemented method of item 1, wherein the one or more solution(s) comprising a nucleic acid cargo and the one or more solution(s) comprising a lipid are provided in one or more multiwell plate(s).
4. The computer-implemented method of item 1, further comprising, prior to step (d), stabilizing the LNPs obtained in step (c) for a particular time.
5. The computer-implemented method of item 1, wherein the one or more LNP preparation(s) are generated in one or more multiwell plate(s).
6. The computer-implemented method of item 1, wherein the nucleic acid is selected from the group consisting of single-stranded DNA, double-stranded DNA, single-stranded RNA, and double-stranded RNA.
7. The computer-implemented method of item 6, wherein the RNA is selected from the group consisting of messenger RNA (mRNA), self-amplifying RNA (saRNA), guide RNA (gRNA), antisense oligonucleotide (ASO), transfer RNA (tRNA), short hairpin RNA (shRNA), circular RNA (circRNA), microRNA (miRNA), and small interfering RNA (siRNA).
8. The computer-implemented method of item 1, wherein the lipid is an ionizable lipid.
9. The computer-implemented method of item 1, wherein the pH in step (c) ranges from pH 2 to pH 6.

10. The computer-implemented method of item 4, wherein the time ranges from 0.1 TO 60 MINUTES.
11. The computer-implemented method of item 1, wherein the pH of the neutralized solution in step (d) ranges from pH 6 to pH 8.
12. The computer-implemented method of item 1, wherein the buffer exchange in step (e) is performed with a buffer, the buffer comprising phosphate-buffered saline (PBS), 4-(2-hydroxyethyl)-1-piperazineethanesulfonic acid (HEPES), or tris(hydroxymethyl)aminomethane (Tris)-buffered saline.
13. The computer-implemented method of item 1, wherein one or more of the particle size, polydispersity, and nucleic acid encapsulation of the one or more LNPs can be measured and/or analyzed by at least one of the following: the Virus Counter® platform (Sartorius Stedim Biotech GmbH, Germany), dynamic light scattering (DLS) and multi-angle light scattering (MALS), single particle automated Raman trapping analysis (SPARTA), high speed imaging technology, PATfix® HPLC platform, Nanoparticle Tracking Analysis, and Forster resonance energy transfer (FRET) assay.
14. The computer-implemented method of item 1, wherein said method further comprises the step of creating a lipid library based on the generated data sets for respective LNP preparations.
15. A computer program product comprising computer-readable instructions, which, when loaded and executed on a computer system, causes the computer system to perform operations according to the method of item 1.
16. A computer-implemented method of monitoring and controlling a process of manufacturing lipid nanoparticles containing nucleic acid cargo,
   wherein said process of manufacturing lipid nanoparticles containing nucleic acid cargo comprises the steps of
   (i) mixing a solution containing the nucleic acid cargo with a lipid-containing solution at an acidic pH;
   (ii) stabilizing the nanoparticles obtained in step (i) for a particular residence time;
   (iii) neutralizing the pH of the mixed solution obtained in step (i) via dilution to further stabilize the nanoparticles obtained in step (ii);
   (iv) performing a buffer exchange on the neutralized solution obtained in step (iii) via ultrafiltration and/or diafiltration (UF/DF) to concentrate the nanoparticles obtained in step (iii) and reformulate the same into a cryogenic buffer;
   said computer-implemented method of monitoring and controlling said process of manufacturing lipid nanoparticles containing nucleic acid cargo comprising the steps of:
   (a) monitoring, during each of steps (i) to (iv), as well as after step (iv), one or more quality parameters, such as particle size, polydispersity, and nucleic acid encapsulation of lipid nanoparticles in real-time;
   (b) determining whether the quality parameters obtained in step (a) during each of steps (i) to (iv), as well as after step (iv), are within predetermined target ranges;
   (c) in case any of the quality parameters obtained in step (a) during each of steps (i) to (iv), as well as after step (iv), are not within said predetermined target ranges, providing feedback control to adjust operating parameters of the respective step in real-time, in order to bring the respective quality parameters into the predetermined target ranges.
17. The computer-implemented method of item 16, wherein the nucleic acid is selected from the group consisting of single-stranded DNA, double-stranded DNA, single-stranded RNA, and double-stranded RNA.

18. The computer-implemented method of item 17, wherein the RNA is selected from the group consisting of messenger RNA (mRNA), self-amplifying RNA (saRNA), guide RNA (gRNA), antisense oligonucleotide (ASO), transfer RNA (tRNA), short hairpin RNA (shRNA), circular RNA (circRNA), microRNA (miRNA), and small interfering RNA (siRNA).
19. The computer-implemented method of item 16, wherein the lipid is an ionizable lipid.
20. The computer-implemented method of item 16, wherein the pH in step (i) ranges from pH 2 to pH 6.
21. The computer-implemented method of item 16, wherein the residence time ranges from 0.5 to 60 minutes.
22. The computer-implemented method of item 16, wherein the pH of the neutralized solution in step (iii) ranges from pH 6 to pH8.
23. The computer-implemented method of item 16, wherein the buffer exchange in step (iv) is performed with a buffer, the buffer comprising phosphate-buffered saline (PBS), 4-(2-hydroxyethyl)-1-piperazineethanesulfonic acid (HEPES), or tris(hydroxymethyl)-aminomethane (Tris)-buffered saline.
24. The computer-implemented method of item 16, wherein the quality parameters comprise one or more of particle size, polydispersity, and nucleic acid encapsulation of lipid nanoparticles and the quality parameters can be measured and/or analyzed by at least one of the following: the Virus Counter® platform (Sartorius Stedim Biotech GmbH, Germany), dynamic light scattering (DLS) and multi-angle light scattering (MALS), single particle automated Raman trapping analysis (SPARTA), high speed imaging technology, PATfix® HPLC platform, Nanoparticle Tracking Analysis, and Forster resonance energy transfer (FRET) assay.
25. The computer-implemented method of item 16, wherein the target range of the particle size is within 5% variation of a specific target particle size value, wherein the target particle size value is between 40 to 400 nm.
26. The computer-implemented method of item 16, wherein the target range of the polydispersity is from 0.04 to 0.1.
27. The computer-implemented method of item 16, wherein the nucleic acid encapsulation has a target range of at least 96% or higher.
28. The computer-implemented method of item 16, wherein the operating parameters that are adjusted in step (c) are selected from the group consisting of nucleic acid flow rate, lipid flow rate, the ratio of nucleic acid flow rate to lipid flow rate, temperature, pressure, nucleation duration, particle growth duration, residence time, maturation period, pH, fluid composition, degree of dilution, dilution rate, mixer type, stream mix ratio, and stirrer/agitator speed.
29. A computer program product comprising computer-readable instructions, which, when loaded and executed on a computer system, causes the computer system to perform operations according to the method of item 16.
30. The computer program product of item 29, wherein said computer-readable instructions comprise control algorithms for the feedback control of operating parameters, wherein said control algorithms are based on mechanistic hybrid models.
31. An automated nanoparticle synthesis system, comprising:
   (a) a first dispenser assembly having a first pump integrated with a first valve;
   (b) a second dispenser assembly having a second pump integrated with a second valve;
   (c) a third valve;
   (d) a microfluidic mixer;
   (e) a lipid reservoir for holding a lipid stock solution;
   (f) a nucleic acid reservoir for holding a nucleic acid stock solution; and
   (g) a waste reservoir, a buffer reservoir, and an ethanol reservoir;
   wherein
   (i) the lipid reservoir is fluidly connected to the first valve and the nucleic acid reservoir is fluidly connected to the second valve, or vice versa;
   (ii) the first valve is fluidly connected to the first pump and the second valve is fluidly connected to the second pump;
   (iii) each of the first valve and the second valve is fluidly connected to the waste reservoir, the buffer reservoir, and the ethanol reservoir, wherein the ports of the first valve and the second valve that are fluidly connected to the waste reservoir, the buffer reservoir, and the ethanol reservoir are different ports from those that are fluidly connected to the lipid reservoir and the nucleic acid reservoir;
   (iv) each of the first valve and the second valve is fluidly connected to the microfluidic mixer; and
   (v) the third valve is fluidly connected to the microfluidic mixer, to the waste reservoir, and to one or more collection reservoir(s) for collecting produced lipid nanoparticles (LNPs).
32. The automated nanoparticle synthesis system of item 31, wherein the first pump and/or the second pump is a syringe pump.
33. The automated nanoparticle synthesis system of item 31, wherein the first valve, the second valve, and/or the third valve is a 6-port valve.
34. The automated nanoparticle synthesis system of item 31, wherein the fluid connections are realized via tubing.
35. The automated nanoparticle synthesis system of item 31, wherein the system further comprises one or more air plugs adapted to separate buffer from RNA-containing solution, one or more air plugs separating buffer from lipid-containing solution, and one or more air plugs separating lipid-containing solution from ethanol.
36. The automated nanoparticle synthesis system of item 31, wherein the microfluidic mixer is a microfluidic mixer chip.
37. An automated nanoparticle synthesis system, comprising:
   (a) a first dispenser assembly having a first pump integrated with a first valve;
   (b) a second dispenser assembly having a second pump integrated with a second valve;
   (c) a microfluidic mixer;
   (d) one or more liquid handlers, wherein said liquid handlers are configured to automate reagent loading and to automate sample collection in one or more well plates; and
   (e) a waste reservoir, a buffer reservoir, and an ethanol reservoir;
   wherein
   (i) the lipid reservoir is fluidly connected to the first valve and the nucleic acid reservoir is fluidly connected to the second valve, or vice versa;
   (ii) the first valve is fluidly connected to the first pump and the second valve is fluidly connected to the second pump;
   (iii) each of the first valve and the second valve is fluidly connected to the waste reservoir, the buffer reservoir, and the ethanol reservoir, wherein the ports of the first valve and the second valve that are fluidly connected to the waste reservoir, the buffer reservoir, and the ethanol reservoir are different ports from those that are fluidly connected to the lipid reservoir and the nucleic acid reservoir;
   (iv) each of the first valve and the second valve is fluidly connected to the microfluidic mixer; and
   (v) said microfluidic mixer is fluidly connected to said liquid handler.
38. The automated nanoparticle synthesis system of item 37, wherein the first pump and/or the second pump is a syringe pump.
39. The automated nanoparticle synthesis system of item 37, wherein the first valve and/or the second valve is a 6-port valve.
40. The automated nanoparticle synthesis system of item 37, comprising a first liquid handler and a second liquid handler, wherein the first liquid handler is configured to automate collection of synthesized LNPs, and the second liquid handler is configured to automate loading of reagents
41. The automated nanoparticle synthesis system of item 37, wherein the fluid connections are realized via tubing.
42. The automated nanoparticle synthesis system of item 37, wherein the system further comprises one or more air plugs adapted to separate buffer from RNA-containing solution, one or more air plugs separating buffer from lipid-containing solution, and one or more air plugs separating lipid-containing solution from ethanol.
43. The automated nanoparticle synthesis system of item 37, wherein the microfluidic mixer is a microfluidic mixer chip.
44. The automated nanoparticle synthesis system of item 37, wherein one or more of said liquid handlers are configured to automatically remove lipid-containing solution and RNA-containing solution from a well plate.

### BRIEF DESCRIPTION OF THE FIGURES

FIG. 1: Exemplary method for the high throughput preparation of lipid nanoparticles (LNPs) containing a nucleic acid cargo.

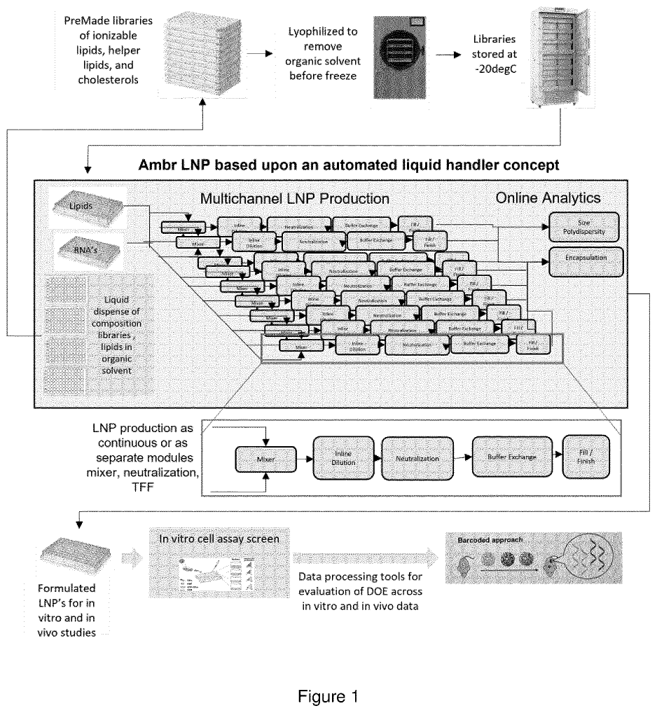

FIG. 2: The process of manufacturing lipid nanoparticles containing nucleic acid cargo of the present invention, wherein the process comprises the steps of mixing, neutralization, buffer exchange, and concentrating the solution containing the product, and wherein the formation of lipid nanoparticles containing nucleic acid cargo is depicted.

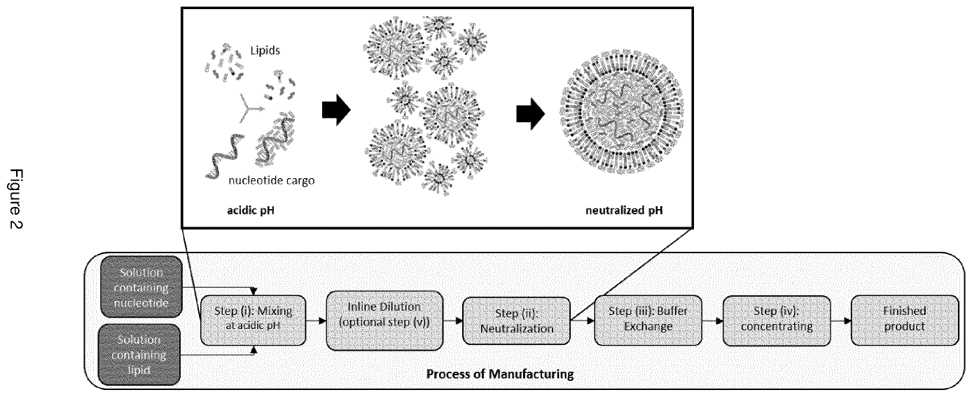

FIG. 3: An embodiment of an automated nanoparticle synthesis system, in accordance with some embodiments.

FIG. 4: An alternative embodiment of an automated nanoparticle synthesis system, in accordance with some embodiments.

FIG. 5: An embodiment of a dispensing module of the automated nanoparticle synthesis system depicted in FIG. 3, in accordance with some embodiments.

FIG. 6: An embodiment of a collection module of the automated nanoparticle synthesis system depicted in FIG. 3, in accordance with some embodiments.

FIG. 7: Demonstration version of the automated nanoparticle synthesis system depicted in FIG. 3, in accordance with some embodiments.

FIG. 8: An embodiment of a user interface of a control software for the automated nanoparticle synthesis systems depicted in FIGS. 3 and 4, in accordance with some embodiments.

FIG. 9: LNP reproducibility. Five different samples of LNPs generated with the automated nanoparticle synthesis system of the present invention showing repeatable size and polydispersity index (PDI).

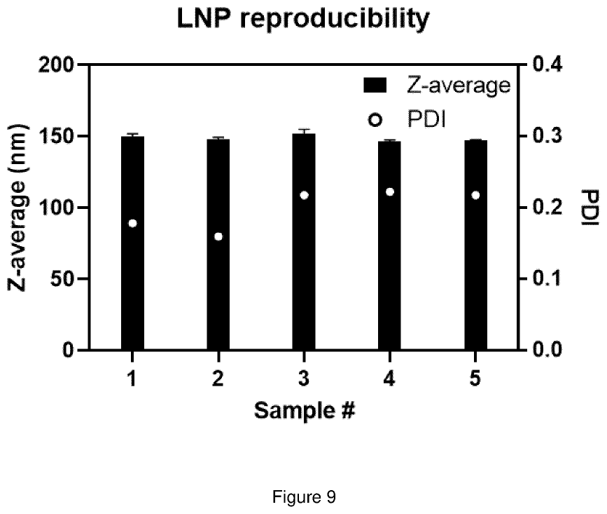

FIG. 10: Effect of flow rate ratios between lipid and mRNA and the total flow rate in the device on size and PDI.

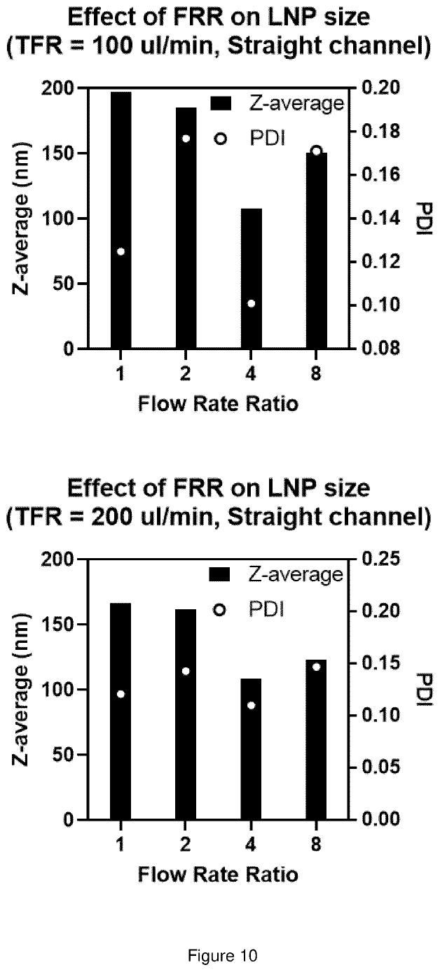

### DETAILED DESCRIPTION OF THE INVENTION

In a first aspect, the present invention relates to a computer-implemented method for the high throughput preparation of lipid nanoparticles (LNPs) containing a nucleic acid cargo, comprising the steps of
(a) providing one or more solution(s) comprising a nucleic acid cargo;
(b) providing one or more solution(s) comprising a lipid;
(c) mixing the one or more solution(s) comprising a nucleic acid cargo with the one or more solution(s) comprising a lipid at an acidic pH;
(d) maturation/stabilization of the LNPs obtained in step (c) for a particular residence time;
(e) neutralizing the pH of the mixed solution obtained in step (c) via dilution to further stabilize the LNPs obtained in step (d);
(f) performing a buffer exchange on the neutralized solution obtained in step (e) via ultrafiltration and/or diafiltration (UF/DF) to concentrate the LNPs obtained in step (e) and reformulate the same into a cryogenic buffer, thereby generating one or more LNP preparation(s);
wherein
during each of steps (c) to (f), as well as after step (f), one or more of the particle size, polydispersity, and nucleic acid encapsulation of the one or more LNPs are monitored in real-time, thereby generating a data set for each LNP preparation; and the generated data set is associated with the respective LNP preparation and stored for further use.

As used herein, the term “computer-implemented method” refers to a method that automatically performed on a computer. Accordingly, the actual preparation of the LNPs, as well as the monitoring and controlling of the process of manufacturing lipid nanoparticles, data set generation, and data set storage is performed automatically by a computer.

In specific embodiments, the nucleic acid cargo is selected from the group consisting of single-stranded DNA, double-stranded DNA, single-stranded RNA, and double-stranded RNA. Further, the RNA can be selected from the group consisting of messenger RNA (mRNA), self-amplifying RNA (saRNA), guide RNA (gRNA), antisense oligonucleotide (ASO), transfer RNA (tRNA), short hairpin RNA (shRNA), circular RNA (circRNA), microRNA (miRNA), and small interfering RNA (siRNA).

In specific embodiments, the lipid used for the production of LNPs in the process of manufacturing LNPs containing nucleic acid cargo is an ionizable lipid. Further, the lipid-containing solution can comprise additional agents, selected from the group consisting of further ionizable lipids, structural lipids, helper lipids (e.g., DSPC (distearoylphosphatidylcholine)), cholesterol, and polyethylene glycol (PEG)-lipid conjugates.

In step (c) of the method of the present invention, one or more solution(s) comprising a nucleic acid cargo are mixed with one or more solution(s) comprising a lipid at an acidic pH, wherein the nucleic acid cargo and the lipid are preferably as defined above. In a preferred embodiment, the nucleic acid cargo is contained in the solution containing the nucleic acid cargo in a concentration of 0.1 to 1 mg/mL. The solvent of the solution containing the nucleic acid cargo is preferably a suitable buffer, e.g., a 20 to 100 mM citrate buffer, pH 3.0. Further, the lipid is preferably contained in the solution containing the lipid in a concentration of 50 to 60 mol %. The solvent of the solution containing the lipid is preferably a suitable organic solvent, e.g., ethanol. Hereby, the volume ratio between the solution containing the nucleic acid cargo and the solution containing the lipid can be from 1:1 to 5:1, preferably about 3:1.

In a specific embodiment, the acidic pH is from pH 2 to pH 6, preferably from pH 3 to pH 6, more preferably from pH 4 to pH 6, and most preferably about pH 5. Moreover, the temperature at which step (c) is performed is not particularly limited, but is preferably room temperature or at a temperature of between room temperature and up to 40° C.

Means of mixing the solution(s) comprising the nucleic acid cargo and the solution(s) comprising the lipid are not particularly limited and are known in the art. However, the mixing is preferably a high-speed laminar mixing, wherein nanoprecipitation is faster than nucleation. Suitable mixers and mixing devices are not particularly limited and are known in the art.

Step (d) of the method of the present invention is a step of maturation and/or stabilization of the LNPs obtained in step (c) by way of maintaining the mixture obtained in step (c) for a certain residence time prior to neutralization in the next step. Suitable residence times can be chosen by the person skilled in the art an amount to e.g., about 0.1 to about 60 minutes.

In step (e) of the method of the present invention, the pH of the mixed solution obtained in step (c) is neutralized via dilution (e.g., inline dilution) to further stabilize the LNPs. In this step, the neutralization is carried out by mixing the mixed solution obtained in step (d) with a solution having a basic pH, preferably phosphate-buffered saline (PBS).

In a preferred embodiment, the mixed solution obtained in the above step (d) is neutralized to a pH ranging from pH 6.0 to pH 8.0, more preferably to a pH ranging from pH 7.0 to pH 8.0, more preferably to a pH ranging from pH 7.2 to pH 7.6, and most preferable to a pH of about pH 7.4.

In step (f) of the method of the present invention, a buffer exchange is performed on the neutralized solution obtained in step (e) via ultrafiltration and/or diafiltration (UF/DF) to concentrate the nanoparticles obtained in step (e) and reformulate the same into a cryogenic buffer. Respective means of performing a buffer exchange are not particularly limited and are known in the art. Suitable cryogenic buffers include for example phosphate buffered saline (PBS), 4-(2-hydroxyethyl)-1-piperazineethanesulfonic acid (HEPES), or tris(hydroxymethyl)aminomethane (Tris)-buffered saline (TBS), containing a suitable cryoprotectant, such as sucrose, mannitol, trehalose, or sorbitol at a concentration of 0.1 to 10 wt. %.

According to the present invention, one or more of the particle size, polydispersity, and nucleic acid encapsulation of the one or more LNPs are monitored during each of steps (c) to (f), as well as after step (f), in real-time, thereby generating a data set for each LNP preparation; and the generated data set is associated with the respective LNP preparation and stored for further use. In specific embodiments, all three of the particle size, polydispersity, and nucleic acid encapsulation of the one or more LNPs are monitored. In other embodiments, only one of said parameters is monitored, or any two of said parameters are monitored.

As used herein, the term “in real-time” refers to the fact that according to the method of the present invention, the above parameters (e.g., quality parameters) of the LNPs prepared in the method of the present invention are monitored while said method is ongoing.

The parameter “particle size” determines the size of the LNPs containing nucleic acid cargo. In a non-limiting embodiment, the particle size is determined by the Virus Counter® platform (Sartorius Stedim Biotech GmbH, Germany), dynamic light scattering (DLS), PATfix® HPLC platform, Nanoparticle Tracking Analysis, and/or multi-angle light scattering (MALS).

The parameter “polydispersity” is a measure of the heterogeneity of the LNPs containing nucleic acid cargo. Polydispersity can occur due to size distribution, agglomeration, or aggregation of the LNPs in solution. In a non-limiting embodiment, the polydispersity is determined by the Virus Counter® platform (Sartorius Stedim Biotech GmbH, Germany), dynamic light scattering (DLS), PATfix® HPLC platform, Nanoparticle Tracking Analysis, multi-angle light scattering (MALS) and/or other suitable tools for measuring particle size distribution, as known in the art.

The parameter “nucleic acid encapsulation” is a measure of the % nucleic acid cargo that is successfully entrapped/adsorbed into the LNPs. In a specific embodiment, the nucleic acid encapsulation composition is determined by the Virus Counter® platform (Sartorius Stedim Biotech GmbH, Germany), Forster resonance energy transfer (FRET) assay, and/or single particle automated Raman trapping analysis (SPARTA).

According to the present invention, a data set is generated for each LNP preparation, i.e., for each of the one or more LNPs that prepared in the method of the present invention. This data set comprises the data obtained by monitoring one or more of the particle size, polydispersity, and nucleic acid encapsulation of the one or more LNPs during each of steps (c) to (f), as well as after step (f). Further, according to the present invention, the respective data set is associated with the respective LNP preparation, i.e., each data set further comprises the information to which LNP preparation it belongs. The data sets are then stored for further use, e.g., to provide feedback regulation of the preparation steps or to enable the evaluation and comparison of the different LNP preparations.

In specific and preferred embodiments of the method of the present invention, two or more individual LNP preparations are generated in parallel. There is no specific upper limit of the number of LNP preparations that can be generated in parallel, other than technical limitations that are imposed by liquid handling and sample handling equipment. In this context, the one or more solution(s) comprising a nucleic acid cargo and/or the one or more solution(s) comprising a lipid can be provided in one or more multiwell plate(s). Suitable multiwell plates are not particularly limited and are known in the art, including e.g., 6-well, 12-well, 24-well, 48-well, 96-well, 384-well and nanowell microtiter plates. In further specific and preferred embodiments, the one or more LNP preparation(s) are also generated in one or more respective multiwell plate(s).

Thus, in preferred embodiments, the method of the present invention provides the possibility of generating two or more LNP preparations in parallel, e.g., 96 LNP preparations in a 96-well plate format. In such embodiments, individual LNP preparations can differ from each other in any way that might be of interest. This includes differences in the solution comprising the nucleic acid (e.g., different nucleic acids, different nucleic acid amounts, different buffer compositions, different pH, different osmolality), differences in the solution comprising the lipid (e.g., different lipids, different structural lipids, different helper lipids, different PEG-lipid conjugates, different buffer compositions, different lipid compositions, different lipid amounts, different pH, different osmolality), and/or differences in the process parameters in steps (c) to (f) (e.g., different mixers, different mixing ratios, different mixing flow rates, different residence times, different buffer compositions, different pH values, different temperatures, different dilution ratios). In this manner, respective LNP preparations can be compared to each other, and the impact of the differing parameters assessed. Further, this allows for a convenient screening method for e.g., nucleic acid libraries, lipid libraries and the like.

Technical means for implementing the method of the present invention are not particularly limited and include robotic liquid handling and laboratory robot system known in the art (e.g., Sartorius Ambr® system).

In specific embodiments, the method of the present invention can be used with lipid libraries to screen for new LNP formulations. Such lipid libraries can be provided in the form of multiwell plates, as indicated above. Further, lipid libraries can be stored and frozen, e.g., in such multiwell plates. In specific embodiments, the lipids can be lyophilized in order to remove organic solvent prior to freezing and storage. In this context, the method of the present invention can represent an integrated approach for lipid library creation. The created lipid library plates can then be used to produce LNPs, the LNPs be analyzed and evaluated in cell assays and animal studies. The results are then used to the dictate the iterative direction of the next formulation runs to be executed using the method of the present invention. This iterative process can result in new, improved LNP formulations.

Further, the method of the present invention can be combined with a screening of the formed LNPs in in vitro and/or in vivo cell assays, optionally followed by animal studies. The results of such assays and studies can influence the next iterations of the design of LNP preparations.

Moreover, the method of the present invention can further comprise a step of creating a lipid library based on the generated data sets for respective LNP preparations.

In specific embodiments, generated datasets from the method of the present invention, e.g. during different iterations of said method, are continuously analyzed by one or more algorithms. In this manner, new, improved LNP formulation opportunities can be identified via approaches, such as reinforcement learning.

In a second aspect, the present invention relates to a computer program product comprising computer-readable instructions, which, when loaded and executed on a computer system, cause the computer system to perform operations according to the method of the present invention. In this aspect, all definitions for the method according to the first aspect of the present invention equally apply.

The computer-implemented method of monitoring and controlling the process of manufacturing LNPs containing nucleic acid cargo of the present invention provides a tool for advantageously monitoring and controlling said process in a robust and reproducible manner from laboratory to industrial scale.

In a third aspect, the present invention relates to a computer-implemented method of monitoring and controlling a process of manufacturing lipid nanoparticles containing nucleic acid cargo.

According to the present invention, said process of manufacturing lipid nanoparticles containing nucleic acid cargo comprises the steps of
(i) mixing a solution containing the nucleic acid cargo with a lipid-containing solution at an acidic pH;
(ii) maturation/stabilization of the nanoparticles obtained in step (i) for a particular residence time;
(iii) neutralizing the pH of the mixed solution obtained in step (i) via dilution to further stabilize the nanoparticles obtained in step (ii);
(iv) performing a buffer exchange on the neutralized solution obtained in step (iii) via ultrafiltration and/or diafiltration (UF/DF) to concentrate the nanoparticles obtained in step (iii) and reformulate the same into a cryogenic buffer;

Further, according to the present invention, said computer-implemented method of monitoring and controlling said process of manufacturing lipid nanoparticles containing nucleic acid cargo comprising the steps of:
(a) monitoring, during each of steps (i) to (iv), as well as after step (iv), one or more quality parameters, such as particle size, polydispersity, and nucleic acid encapsulation of lipid nanoparticles in real-time;
(b) determining whether the quality parameters obtained in step (a) during each of steps (i) to (iv), as well as after step (iv), are within predetermined target ranges;
(c) in case any of the quality parameters obtained in step (a) during each of steps (i) to (iv), as well as after step (iv), are not within said predetermined target ranges, providing feedback control to adjust operating parameters of the respective step in real-time, in order to bring the respective quality parameters into the predetermined target ranges.

In specific embodiments, the nucleic acid cargo is selected from the group consisting of single-stranded DNA, double-stranded DNA, single-stranded RNA, and double-stranded RNA. Further, the RNA can be selected from the group consisting of messenger RNA (mRNA), self-amplifying RNA (saRNA), guide RNA (gRNA), antisense oligonucleotide (ASO), transfer RNA (tRNA), short hairpin RNA (shRNA), circular RNA (circRNA), microRNA (miRNA), and small interfering RNA (siRNA).

In specific embodiments, the lipid used for the production of lipid nanoparticles in the process of manufacturing lipid nanoparticles containing nucleic acid cargo is an ionizable lipid. Further, the lipid-containing solution can comprise additional agents, selected from the group consisting of further ionizable lipids, structural lipids, helper lipids (e.g. DSPC(distearoylphosphatidylcholine)), cholesterol, and polyethylene glycol (PEG)-lipid conjugates.

In step (i) of the process of manufacturing lipid nanoparticles containing nucleic acid cargo, a solution containing the nucleic acid cargo is mixed with a solution containing a lipid at an acidic pH, wherein the nucleic acid cargo and the lipid are preferably as defined above. In a preferred embodiment, the nucleic acid cargo is contained in the solution containing the nucleic acid cargo in a concentration of 0.1 to 1 mg/mL. The solvent of the solution containing the nucleic acid cargo is preferably a suitable buffer, e.g. a 20 to 100 mM citrate buffer, pH 3.0.

Further, the lipid is preferably contained in the solution containing the lipid in a concentration of 50 to 60 mol %. The solvent of the solution containing the lipid is preferably a suitable organic solvent, e.g. ethanol.

Hereby, the volume ratio between the solution containing the nucleic acid cargo and the solution containing the lipid can be from 1:1 to 5:1, preferably about 3:1.

In a specific embodiment, the acidic pH is from pH 2 to pH 6, preferably from pH 3 to pH 6, more preferably from pH 4 to pH 6, and most preferably about pH 5. Moreover, the temperature of step (i) is not particularly limited, but step (i) is preferably carried out at room temperature or at a temperature of between room temperature and up to 40° C.

Means of mixing the solution containing the nucleic acid cargo and the solution containing the lipid are not particularly limited and are known in the art. However, the mixing is preferably a high-speed laminar mixing, wherein nanoprecipitation is faster than nucleation.

Step (ii) of the process of manufacturing lipid nanoparticles containing nucleic acid cargo is a step of maturation and/or stabilization of the lipid nanoparticles obtained in step (i) by way of maintaining the mixture obtained in step (i) for a certain residence time prior to neutralization in the next step. Suitable residence times can be chosen by the person skilled in the art an amount to e.g. about 0.5 to about 60 minutes.

In step (iii) of the process of manufacturing lipid nanoparticles containing nucleic acid cargo, the pH of the mixed solution obtained in step (i) is neutralized via dilution (e.g., inline dilution) to further stabilized the lipid nanoparticles. In this step, the neutralization is carried out by mixing the mixed solution obtained in step (i) with a solution having a basic pH, preferably phosphate-buffered saline (PBS).

In a preferred embodiment, the mixed solution obtained in the above step (i) is neutralized to a pH ranging from pH 6.0 to pH 8.0, more preferably to a pH ranging from pH 7.0 to pH 8.0, more preferably to a pH ranging from pH 7.2 to pH 7.6, and most preferable to a pH of about pH 7.4.

In step (iv) of the process of manufacturing lipid nanoparticles containing nucleic acid cargo, a buffer exchange is performed on the neutralized solution obtained in step (iii) via ultrafiltration and/or diafiltration (UF/DF) to concentrate the nanoparticles obtained in step (iii) and reformulate the same into a cryogenic buffer. Respective means of performing a buffer exchange are not particularly limited and are known in the art. Suitable cryogenic buffers include for example phosphate buffered saline (PBC) containing a suitable cryoprotectant, such as sucrose, mannitol, trehalose, or sorbitol at a concentration of 0.1 to 10 wt %.

In a specific embodiment, the process of manufacturing lipid nanoparticles containing nucleic acid cargo further comprises a step (ii-a), which is carried out between the above steps (ii) and (iii). In step (ii-a), the solution obtained in the above step (ii) is diluted before the diluted solution is neutralized in step (iii). Hereby, the mixed solution obtained in the above step (i) is diluted with a solution comprising 20 to 30% of an alcohol, preferably ethanol. The mixed solution is diluted 2 to 4 times based on the volume before the dilution.

In step (a) of the method of the present invention, the quality parameters, such as particle size, polydispersity, and nucleic acid encapsulation of lipid nanoparticles are monitored during each of steps (i) to (iv), as well as after step (iv) in real-time. In specific embodiments, all three of the above quality parameters are monitored. In other embodiments, only one of said quality parameters is monitored, or any combination of two of said quality parameters is monitored.

In step (b) of the method of the present invention, it is determined whether the quality parameters obtained in step (a) during each of steps (i) to (iv), as well as after step (iv), are within predetermined target ranges.

Further, in step (c) of the method of the present invention, in case any of the quality parameters obtained in step (a) during each of steps (i) to (iv), as well as after step (iv), of the process of manufacturing lipid nanoparticles containing nucleic acid cargo are not within said predetermined target ranges, feedback control is provided to adjust operating parameters in real-time, in order to bring the respective quality parameters into the predetermined target ranges.

As used herein, the term “feedback control to adjust operating parameters” refers to feedback as to whether the target ranges of the quality parameters are met or whether the measured values are above or below the target ranges. Based thereon, the operating parameters in each of the steps (i) to (iv) in the process of manufacturing can be adjusted in order to bring the respective quality parameter into the target range.

In specific embodiments, the operating parameters that are adjusted in step (c) of the method of the present invention are selected from the group consisting of nucleic acid flow rate, lipid flow rate, the ratio of nucleic acid flow rate to lipid flow rate, temperature, pressure, nucleation duration, particle growth duration, residence time, maturation period, pH, fluid composition, degree of dilution, dilution rate, mixer type, stream mix ratio, and stirrer/agitator speed.

The quality parameter “particle size” determines the size of the lipid nanoparticles containing nucleic acid cargo. The target range of the particle size is within 5% variation of a specific target particle size value, wherein the target particle size value can be between 40 to 400 nm, depending on the intended therapeutic application. In a non-limiting embodiment, the particle size is determined by the Virus Counter® platform (Sartorius Stedim Biotech GmbH, Germany), dynamic light scattering (DLS), PATfix® HPLC platform, Nanoparticle Tracking Analysis, and/or multi-angle light scattering (MALS).

If the particle size is above or below the target range, one or more of the above operating parameters are adjusted in order to decrease or increase the particle size to the predetermined target range.

The quality parameter “polydispersity” is a measure of the heterogeneity of the lipid nanoparticles containing nucleic acid cargo. Polydispersity can occur due to size distribution, agglomeration, or aggregation of the lipid nanoparticles in solution. The target range of the particle size is preferably from 0.04 to 0.1. In a non-limiting embodiment, the polydispersity is determined by the Virus Counter® platform (Sartorius Stedim Biotech GmbH, Germany), dynamic light scattering (DLS), PATfix® HPLC platform, Nanoparticle Tracking Analysis, multi-angle light scattering (MALS) and/or other suitable tools for measuring particle size distribution, as known in the art.

If the polydispersity is above or below the target range, one or more of the above operating parameters are adjusted in order to decrease or increase the polydispersity to the predetermined target range.

The quality parameter “nucleic acid encapsulation” is a measure of the % nucleic acid cargo that is successfully entrapped/adsorbed into the lipid nanoparticles. The target range of the nucleic acid encapsulation composition is preferably at least 96% or higher. In a specific embodiment, the nucleic acid encapsulation composition is determined by the Virus Counter® platform (Sartorius Stedim Biotech GmbH, Germany), Förster resonance energy transfer (FRET) assay, and/or single particle automated Raman trapping analysis (SPARTA).

If the nucleic acid encapsulation composition is above or below the target range, one or more of the above operating parameters are adjusted in order to increase the nucleic acid encapsulation composition to the predetermined target range.

In a specific embodiment, the pressure is measured in steps (i) and (iv) of the process of manufacturing. A pressure that is above the target range represents an indication of potential fouling.

In another specific embodiment, the pH is measured in steps (i) and (ii) of the manufacturing process. The target ranges of the pH are as described above.

In a fourth aspect, the present invention relates to a computer program product comprising computer-readable instructions, which, when loaded and executed on a computer system, cause the computer system to perform operations according to the method of the present invention. In this aspect, all definitions for the method according to the first aspect of the present invention equally apply.

In specific embodiments, said computer-readable instructions comprise control algorithms for the feedback control of the above operating parameters, wherein said control algorithms are based on mechanistic hybrid models.

The computer-implemented method of monitoring and controlling the process of manufacturing lipid nanoparticles containing nucleic acid cargo of the present invention provides a tool for advantageously monitoring and controlling said process in a robust and reproducible manner from laboratory to industrial scale.

In this context, the present invention encompasses, in specific embodiments, the creation and use of algorithms to optimize the control of LNP manufacture. Specifically, algorithms may be used to control the impact of the values associated with different operating parameters (e.g., nucleic acid flow rate, lipid flow rate, the ratio of nucleic acid flow rate to lipid flow rate, temperature, pressure, nucleation duration, particle growth duration, residence time, maturation period, pH, fluid composition, degree of dilution, dilution rate, mixer type, stream mix ratio, and stirrer/agitator speed) to maximize performance of the LNPs. Continuous improvements of LNP manufacturing robustness will be applied by continued training of the algorithm models by techniques such as reinforcement learning. a fifth aspect, the present invention relates to an automated nanoparticle synthesis system, comprising:
(a) two dispensers, said dispensers each comprising a syringe pump and a 6-port valve, wherein said syringe pump is fluidly connected to the input port of said 6-port valve;
(b) a 6-port distribution valve;
(c) a microfluidic mixer chip;
(d) a waste reservoir, a buffer reservoir, and an ethanol reservoir;
wherein
(i) said dispensers are both fluidly connected via the 6-port valve to said waste reservoir, said buffer reservoir, and said ethanol reservoir; and to said microfluidic mixer chip;
(ii) the first of said dispenser is further fluidly connected via the 6-port valve to a lipid supply;
(iii) the second of said dispenser is further fluidly connected via the 6-port valve to an RNA supply;
(iv) said microfluidic mixer chip is fluidly connected to the input port of said distribution valve; and
(v) said distribution valve is fluidly connected to said waste reservoir; and to lipid nanoparticle (LNP) collection tubes.

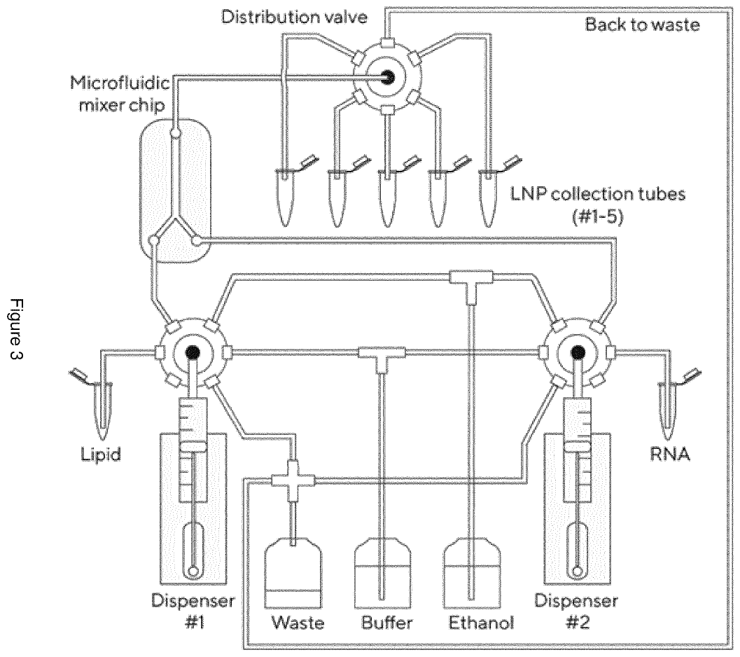

The automated nanoparticle synthesis system of the present invention comprises a dispenser assembly having a first pump (e.g., syringe pump) integrated with a valve (e.g., a 6-port valve) and a second dispenser assembly having a second pump (e.g., syringe pump) integrated with a second valve (e.g., a 6-port valve). The system also comprises one 6-port distribution valve. A microfluidic mixer is utilized achieve fast and controllable mixing of lipid and RNA for the generation of LNPs (FIG. 3). The automated system aims at increasing the throughput of LNP screening activities for different microfluidic mixer designs and key flow parameters affecting quality of LNPs, such as FRR (flow rate ratio) and TFR (total flow rate).

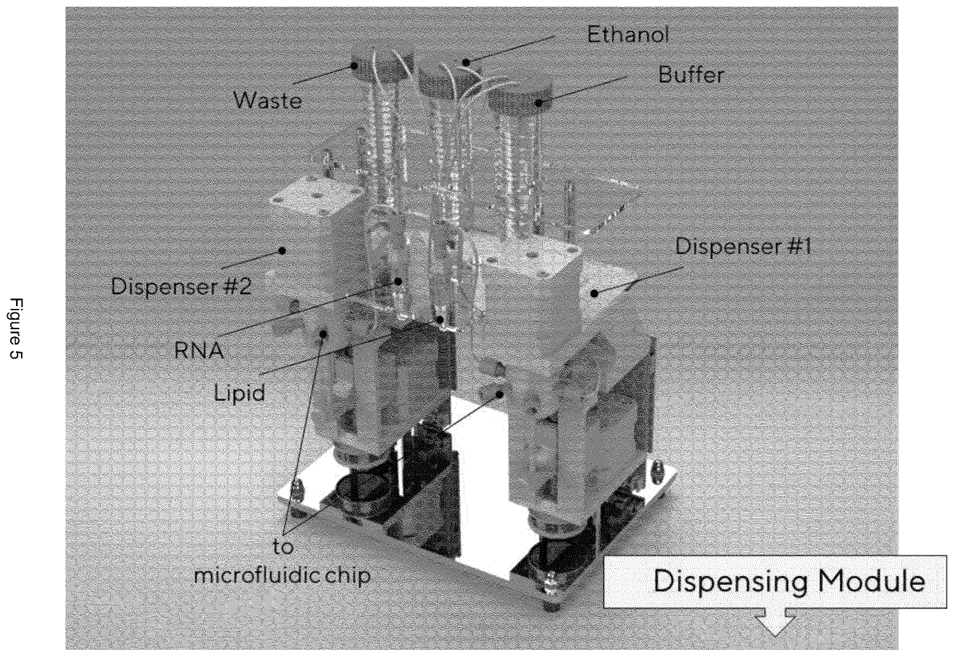

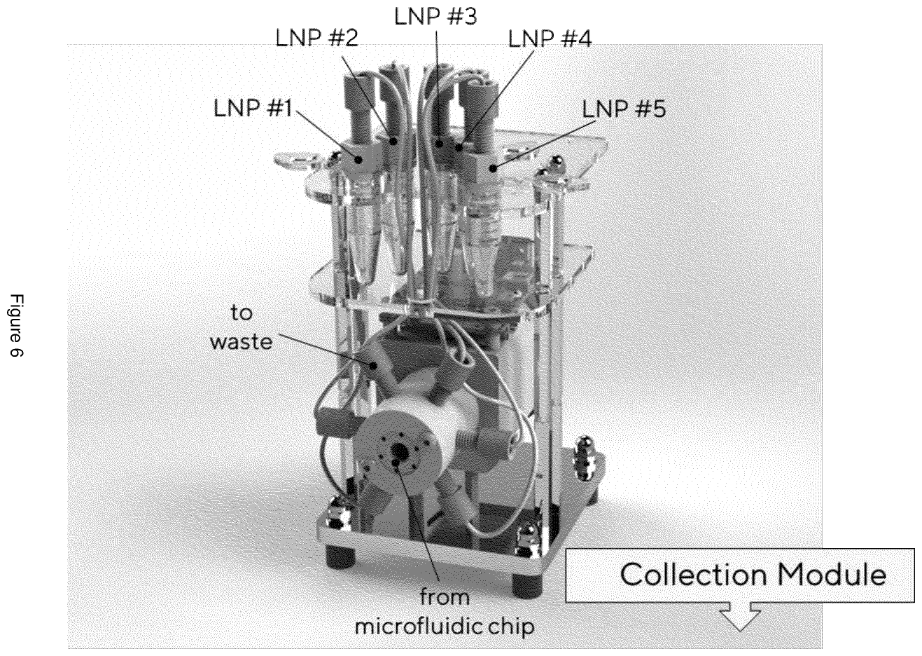

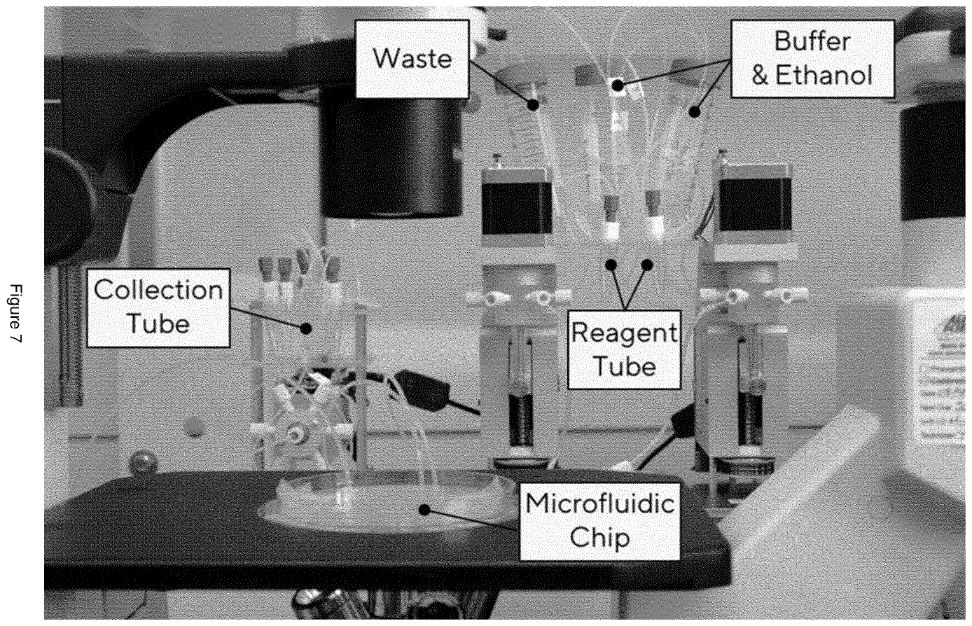

The automated nanoparticle synthesis system of the present invention can be divided into (i) a dispensing module for loading and dispensing reagents and pushing liquids to the microfluidic mixing assembly (e.g., microfluidic mixing chip) chip located downstream (FIG. 5), and (ii) a collection module for collecting the synthesized LNPs under different screening conditions and storing them (FIG. 6). FIG. 7 shows a demonstration version of the automated nanoparticle synthesis system of the present invention.

As seen in FIG. 5 and as a non-limiting example, the dispensing module comprises a lipid reservoir for holding a lipid stock solution and a nucleic acid reservoir for holding an aqueous nucleic acid stock solution, such as RNA stock solution. These reservoirs may be commercially available reservoirs, such as tubes, wells, or bags. In other examples, reservoirs may hold other nucleic acid stock solutions, such as DNA, miRNA, cDNA, etc.

The first reservoir is fluidly connected to the first valve (e.g., a 6-port valve) on the first dispenser and the second reservoir is fluidly connected to the second valve (e.g., a 6-port valve) on the second dispenser. The first valve is fluidically coupled to the first pump (e.g., syringe pump) and the second valve is fluidically coupled to the second pump (e.g., syringe pump). Each of the first valve and the second valve may be commercially available valves. In addition to syringe pumps, each of the first pump and the second pump may be any other suitable pumps, such as peristaltic pumps, centrifugal pumps, membrane pumps, and/or piston pumps in other embodiments.

As seen in FIG. 5, each of the first valve and the second valve is fluidically coupled (via separate ports on the first valve and the second valve and via tubing) to the waste reservoir, the buffer reservoir, and the ethanol reservoir. The ports on the first vale and the second valve that are fluidically coupled to the waste reservoir, the buffer reservoir, and the ethanol reservoir are different ports from those fluidically coupled to the lipid reservoir and the nucleic acid reservoir. Each of the first valve and the second valve are configured to automatically switch among the buffer reservoir, the ethanol reservoir, the lipid reservoir, and/or the nucleic acid reservoir. These reservoirs may be commercially available reservoirs, such as tubes, wells, or bags.

As seen in FIG. 5, each of the first and second valves is fluidically coupled, via tubing, to the mixing assembly (e.g., microfluidic mixer chip) . The mixing assembly may be used for in-line mixing of the lipid stock solution and the aqueous nucleic acid solution use precise flow control of the two stock solutions to form a dilute intermediate product.

As seen in FIGS. 3 and 6 and as a non-limiting example of the collection module, the distribution valve (e.g., a 6-port valve) is fluidically coupled, via tubing, to the mixing assembly (e.g., microfluidic mixer chip). One port of the distribution valve is fluidically coupled, via tubing, to the waste reservoir and other individual ports of the distribution valve are fluidically coupled, via tubing, to one or more lipid nanoparticle (LNP) collection tubes for collecting and storing synthesized lipid nanoparticles produced from the mixture of the two stock solutions. The distribution valve directs the flow of waste to the waste reservoir while controlling the flow of formulated lipid nanoparticles into the LNP collection tubes. Even though FIGS. 3 and 6 show 5 LNP collection tubes, there may either be less than 5 or more than 5 LNP collection tubes in other embodiments.

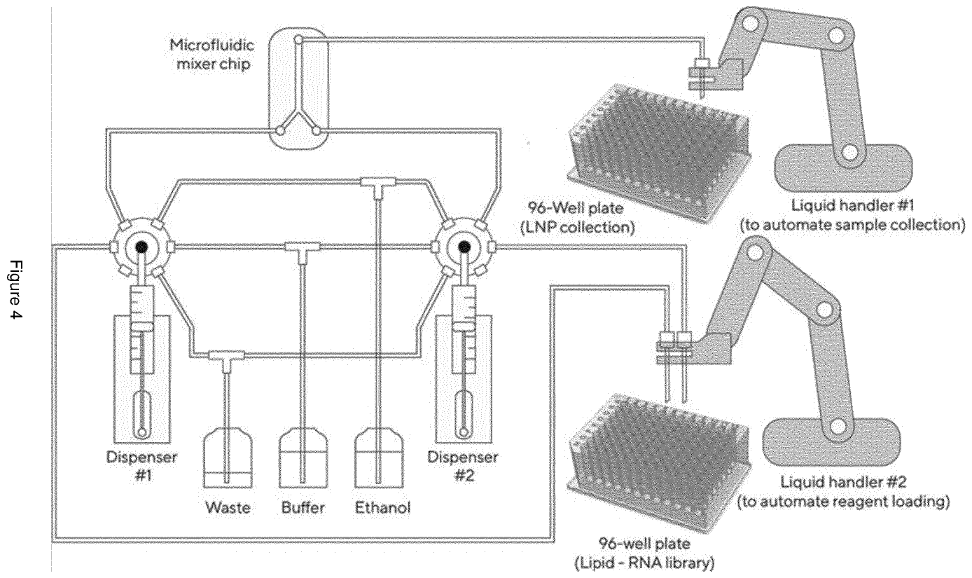

FIG. 4 shows an alternative embodiment of an automated nanoparticle synthesis system. As with the system shown in FIG. 3, the system shown in FIG. 4 also comprises a first dispenser assembly having a first pump (e.g., syringe pump) integrated with a first valve (e.g., a 6-port valve) and a second dispenser assembly having a second pump (e.g., syringe pump) integrated with a second valve (e.g., a 6-port valve).

As with the system shown in FIG. 3, the system shown in FIG. 4 also comprises a waste reservoir, a buffer reservoir, and an ethanol reservoir, wherein each of the first valve and the second valve is fluidically coupled to the waste reservoir, the buffer reservoir, and the ethanol reservoir via separate ports on the valves. Each of the first and the second valves is also fluidically coupled to a mixing assembly (e.g., a microfluidic mixer chip) to provide fast and controllable mixing of lipid and RNA for the generation of LNPs.

Unlike the system shown in FIG. 3, the system shown in FIG. 4 does not include a distribution valve for sample collection. Instead, the system shown in FIG. 4 comprises a first liquid handler configured to automate collection of synthesized LNPs, and a second liquid handler configured to automate loading of reagents for many different combinations, such as lipid stock solution and RNA stock solution, in a well plate. In the example shown in FIG. 4, the first liquid handler is configured to automatically transfer synthesized LNPs into a well plate (e.g., 96-well plate) for collection and storage. The second liquid handler is configured to remove lipid stock solution and nucleic acid stock solution from a well plate (e.g., 96-well plate). For example, the well plate may have a lipid-RNA library, wherein lipid stock solution is present in one or more wells and RNA stock solution is present in one or more wells that do not include lipid stock solution. Other embodiments may have just 1 liquid handler configured to both automate collection of synthesized LNPs and loading of reagents.

The screening throughput and capability of the system shown in FIG. 3 can be further improved by integrating extra well plates and one or more liquid handlers in the system shown in FIG. 4. As a result, the system shown in FIG. 4 does not require sample injections, which allows for the use of smaller sample amounts.

Any of the systems and components thereof described herein, including the various pumps and valves described herein, can be controlled using any known control techniques and/or any known control systems. For example, in some embodiments, such components can be controlled manually, electronically, and/or hydraulically. In the system shown in FIG. 3 up to five combinations of TFR and FRR (from 1 to 8) can be used in one automatic run. Further, screening flow rates in the range of about 5 μL/min to 30 mL/min can be used for each phase (aqueous/organic). Furthermore, the systems described herein allow for the use of sample volumes as small as 100 UL.

The amount of buffer and ethanol used in the automated nanoparticle synthesis systems described here depends on several factors, including: (i) the length and inner diameter of tubing defining the fluid path that connects the pumps (e.g., syringe pumps), the microfluidic mixer chip, the distribution valve, and the collection reservoirs; (ii) the number of screenings performed; and (iii) the number of cycles for washing (by ethanol) and priming (by buffer) the systems. For example, where the total length of tubing is 4000 mm and its inner diameter is 0.8 mm, the amount of buffer and ethanol required for performing one individual screening and one cycle of washing and priming is typically within 5 mL.

In a sixth aspect, the present invention relates to the use of one or more air plugs in the systems described herein to separate the buffer, the lipid-containing solution, the RNA-containing solution, and/or the ethanol solution from each other in the tubing lines that provide the fluidic connections between the components.

An air plug is an amount of air that is introduced into the tubing lines in order to provide separation between the buffer, the lipid-containing solution, the RNA-containing solution, and/or the ethanol solution. For example, a microfluidic mixer chip may include air plugs separating RNA solution from buffer and air plugs separating buffer from lipid solution and lipid solution from ethanol solution. In an embodiment, the volume of the air plug is between 1 μL and 20 μL, preferably about 5 82 L.

Without air plugs, RNA and lipids can diffuse into their surrounding fluid (e.g., buffer and ethanol) within a tubing. Such unwanted diffusion can change the RNA concentration and the lipid/RNA mixing ratio at the time of particle synthesis through a microfluidic mixer. In preliminary experiments without the use of air plugs, RNA was found in waste samples, while RNA was found in mostly samples with the use of air plugs. As a result, air plugs are particularly important for preparing samples having small volumes while minimizing the waste of reagents.

An embodiment of a related LNP screening workflow according to the present disclosure can involve the following steps (a) to (f):
(a) adding an air plug into the tubing lines of a nanoparticle synthesis system;
(b) loading lipid and RNA into a sample loop of said system;
(c) adding another air plug into the tubing lines of said system;
(d) pushing lipid and RNA to meet at a flow focusing junction of said system;
(e) coordinating the flow to generate LNPs; and
(f) switching a valve of said system to collect LNPs.

In this workflow, the nanoparticle synthesis system is preferably the automated nanoparticle synthesis system shown in FIG. 3 of the present disclosure.

In some embodiments, LNP screening based on the above-mentioned methods occurs via a slow protocol involving microfluidic chip cleaning and priming steps between screening tasks. In alternative embodiments LNP screening based on the above-mentioned methods occurs via a fast protocol without cleaning and priming steps between screening tasks.

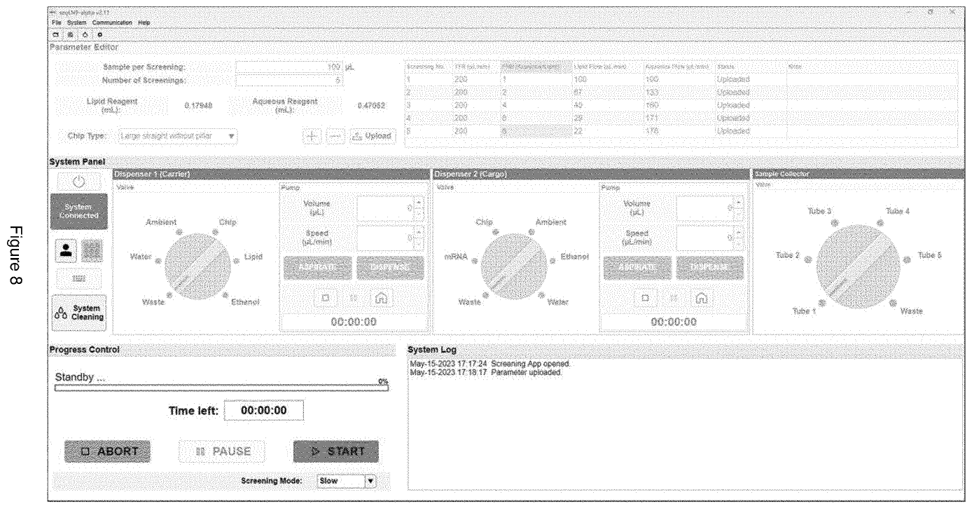

FIG. 8 shows an exemplary user interface of a control software that can be used with the automated nanoparticle synthesis systems of the present disclosure. This software provides an easy-to-use interface, showing e.g. user-chosen flow rate ratio (FFR), total flow rate (TFR), and sample volume. Different microfluidic chip designs can be added to a library for future screening. An advanced manual mode enables more flexibility for the use to control every device in the system.

As used herein, the term “comprising”/“comprises” expressly includes the terms “consisting essentially of”/“consists essentially of” and “consisting of”/“consists of”, i.e., all of the terms are interchangeable with each other herein.

Further, as used herein, the term “about” preferably represents a modifier of the specified value of ±10%, more preferably ±8%, ±6%, ±5%, ±4%, ±3%, ±2%, ±1%, or ±0.5%. Thus, by way of example, the term “about 100” can include the ranges of 90 to 110, 92 to 108, 94 to 106, 95 to 105, 96 to 104, 97 to 103, 98 to 102, 99 to 101, or 99.5 to 100.5.

The present invention advantageously provides a method for the high-throughput preparation of LNPs containing a nucleic acid cargo, affording the preparation of hundreds of different LNP preparations in parallel per person per day. This is combined with monitoring important quality parameters (e.g., particle size, polydispersity, nucleic acid encapsulation) online and in real-time. Accordingly, the present invention provides fast and easy to use means for generating, screening, and analyzing LNPs in a high-throughput format.

The present invention according to the fifth aspect will be further illustrated in the following example without any limitation thereto.

### EXAMPLE

1,2-Dioleoyl-3-trimethylammonium propane (DOTAP) blank LNP formulation was prepared using the automated nanoparticle synthesis systems disclosed herein. DOTAP blank LNP formulation includes 4 lipids, DOTAP, Distearoylphosphatidylcholine (DSPC), cholesterol, and DMG-PEG 2000. The molar ratio of these lipids is 40:10:48:2. Lipid was dissolved in ethanol having a concentration of about 7.5 mM and citric buffer having pH 4 and a concentration of about 10 mM as an aqueous phase. In this example, LNP production scale is 200 uL. Five LNP samples were produced continuously. Flow rate ratio (FRR) was 2 and total flow rate (TFR) was 200 μL/min.

The microfluidics chip and production system were cleaned with 10 mM citric buffer and ethanol after every sample production. Particle size (Z-average) and polydispersity index (PDI) were measured using Zetasizer Pro (Malvern).

Effect of flow rate ratios between the lipid and mRNA and the total flow rate in the device on size and polydispersity index was studied. The same DOTAP formulation was prepared. Different FRR (1, 2, 4, 8) and TFR (100 and 200 μL/min) was used for mixing. For this study, the LNP production volume was 100 μL, and the final lipid concentration was 2.5 mM. The microfluidics chip and production system were cleaned with 10 mM citric buffer and ethanol after every sample production. Particle size (Z-average) and polydispersity index (PDI) were measured using Zetasizer Pro (Malvern).

1. A computer-implemented method for the high throughput preparation of lipid nanoparticles (LNPs) containing a nucleic acid cargo, comprising the steps of
(a) providing one or more solution(s) comprising a nucleic acid cargo;
(b) providing one or more solution(s) comprising a lipid;
(c) mixing the one or more solution(s) comprising a nucleic acid cargo with the one or more solution(s) comprising a lipid at an acidic pH;
(d) neutralizing the pH of the mixed solution obtained in step (c) via dilution to stabilize the LNPs obtained in step (c);
(e) performing a buffer exchange on the neutralized solution obtained in step (d) via ultrafiltration and/or diafiltration (UF/DF) to concentrate the LNPs obtained in step (d) and reformulate the same into a cryogenic buffer, thereby generating one or more LNP preparation(s);
wherein
during each of steps (c) to (e), as well as after step (e), one or more of the particle size, polydispersity, and nucleic acid encapsulation of the one or more LNPs are monitored in real-time, thereby generating a data set for each LNP preparation; and the generated data set is associated with the respective LNP preparation.

2. The computer-implemented method of claim 1, wherein two or more individual LNP preparations are generated in parallel.

3-8. (canceled)

9. The computer-implemented method of claim 1, wherein the pH in step (c) ranges from pH 2 to pH 6.

10-11. (canceled)

12. The computer-implemented method of claim 1, wherein the buffer exchange in step (e) is performed with a buffer, the buffer comprising phosphate-buffered saline (PBS), 4-(2-hydroxyethyl)-1-piperazineethanesulfonic acid (HEPES), or tris(hydroxymethyl)aminomethane (Tris)-buffered saline.

13. The computer-implemented method of claim 1, wherein one or more of the particle size, polydispersity, and nucleic acid encapsulation of the one or more LNPs can be measured and/or analyzed by at least one of the following: the Virus Counter® platform (Sartorius Stedim Biotech GmbH, Germany), dynamic light scattering (DLS) and multi-angle light scattering (MALS), single particle automated Raman trapping analysis (SPARTA), high speed imaging technology, PATfix® HPLC platform, Nanoparticle Tracking Analysis, and Förster resonance energy transfer (FRET) assay.

14. The computer-implemented method of claim 1, wherein said method further comprises the step of creating a lipid library based on the generated data sets for respective LNP preparations.

15. (canceled)

16. A computer-implemented method of monitoring and controlling a process of manufacturing lipid nanoparticles containing nucleic acid cargo,
wherein said process of manufacturing lipid nanoparticles containing nucleic acid cargo comprises the steps of
(i) mixing a solution containing the nucleic acid cargo with a lipid-containing solution at an acidic pH;
(ii) stabilizing the nanoparticles obtained in step (i) for a particular residence time;
(iii) neutralizing the pH of the mixed solution obtained in step (i) via dilution to further stabilize the nanoparticles obtained in step (ii);
(iv) performing a buffer exchange on the neutralized solution obtained in step (iii) via ultrafiltration and/or diafiltration (UF/DF) to concentrate the nanoparticles obtained in step (iii) and reformulate the same into a cryogenic buffer;
said computer-implemented method of monitoring and controlling said process of manufacturing lipid nanoparticles containing nucleic acid cargo comprising the steps of:
(a) monitoring, during each of steps (i) to (iv), as well as after step (iv), one or more quality parameters, such as particle size, polydispersity, and nucleic acid encapsulation of lipid nanoparticles in real-time;
(b) determining whether the quality parameters obtained in step (a) during each of steps (i) to (iv), as well as after step (iv), are within predetermined target ranges;
(c) in case any of the quality parameters obtained in step (a) during each of steps (i) to (iv), as well as after step (iv), are not within said predetermined target ranges, providing feedback control to adjust operating parameters of the respective step in real-time, in order to bring the respective quality parameters into the predetermined target ranges.

17-24. (canceled)

25. The computer-implemented method of claim 16, wherein the target range of the particle size is within 5% variation of a specific target particle size value, wherein the target particle size value is between 40 to 400 nm.

26. The computer-implemented method of claim 16, wherein the target range of the polydispersity is from 0.04 to 0.1.

27. The computer-implemented method of claim 16, wherein the nucleic acid encapsulation has a target range of at least 96% or higher.

28. The computer-implemented method of claim 16, wherein the operating parameters that are adjusted in step (c) are selected from the group consisting of nucleic acid flow rate, lipid flow rate, the ratio of nucleic acid flow rate to lipid flow rate, temperature, pressure, nucleation duration, particle growth duration, residence time, maturation period, pH, fluid composition, degree of dilution, dilution rate, mixer type, stream mix ratio, and stirrer/agitator speed.

29. A computer program product comprising computer-readable instructions, which, when loaded and executed on a computer system, cause the computer system to perform operations according to the method of claim 16, wherein said computer-readable instructions comprise control algorithms for the feedback control of operating parameters, and wherein said control algorithms are based on mechanistic hybrid models.

30. (canceled)

31. An automated nanoparticle synthesis system, comprising:
(a) a first dispenser assembly having a first pump integrated with a first valve;
(b) a second dispenser assembly having a second pump integrated with a second valve;
(c) a third valve;
(d) a microfluidic mixer;
(e) a lipid reservoir for holding a lipid stock solution;
(f) a nucleic acid reservoir for holding a nucleic acid stock solution; and
(g) a waste reservoir, a buffer reservoir, and an ethanol reservoir;
wherein
(i) the lipid reservoir is fluidly connected to the first valve and the nucleic acid reservoir is fluidly connected to the second valve, or vice versa;
(ii) the first valve is fluidly connected to the first pump and the second valve is fluidly connected to the second pump;
(iii) each of the first valve and the second valve is fluidly connected to the waste reservoir, the buffer reservoir, and the ethanol reservoir, wherein the ports of the first valve and the second valve that are fluidly connected to the waste reservoir, the buffer reservoir, and the ethanol reservoir are different ports from those that are fluidly connected to the lipid reservoir and the nucleic acid reservoir;
(iv) each of the first valve and the second valve is fluidly connected to the microfluidic mixer; and
(v) the third valve is fluidly connected to the microfluidic mixer, to the waste reservoir, and to one or more collection reservoir(s) for collecting produced lipid nanoparticles (LNPs).

32. The automated nanoparticle synthesis system of claim 31, wherein the first pump and/or the second pump is a syringe pump.

33. The automated nanoparticle synthesis system of claim 31, wherein the first valve, the second valve, and/or the third valve is a 6-port valve.

34. (canceled)

35. The automated nanoparticle synthesis system of claim 31, wherein the system further comprises one or more air plugs adapted to separate buffer from RNA-containing solution, one or more air plugs separating buffer from lipid-containing solution, and one or more air plugs separating lipid-containing solution from ethanol.

36. The automated nanoparticle synthesis system of claim 31, wherein the microfluidic mixer is a microfluidic mixer chip.

37. An automated nanoparticle synthesis system, comprising:
(a) a first dispenser assembly having a first pump integrated with a first valve;
(b) a second dispenser assembly having a second pump integrated with a second valve;
(c) a microfluidic mixer;
(d) one or more liquid handlers, wherein said liquid handlers are configured to automate reagent loading and to automate sample collection in one or more well plates; and
(e) a waste reservoir, a buffer reservoir, and an ethanol reservoir;
wherein
(i) the lipid reservoir is fluidly connected to the first valve and the nucleic acid reservoir is fluidly connected to the second valve, or vice versa;
(ii) the first valve is fluidly connected to the first pump and the second valve is fluidly connected to the second pump;
(iii) each of the first valve and the second valve is fluidly connected to the waste reservoir, the buffer reservoir, and the ethanol reservoir, wherein the ports of the first valve and the second valve that are fluidly connected to the waste reservoir, the buffer reservoir, and the ethanol reservoir are different ports from those that are fluidly connected to the lipid reservoir and the nucleic acid reservoir;
(iv) each of the first valve and the second valve is fluidly connected to the microfluidic mixer; and
(v) said microfluidic mixer is fluidly connected to said liquid handler.

38-39. (canceled)

40. The automated nanoparticle synthesis system of claim 37, comprising a first liquid handler and a second liquid handler, wherein the first liquid handler is configured to automate collection of synthesized LNPs, and the second liquid handler is configured to automate loading of reagents

41-43. (canceled)

44. The automated nanoparticle synthesis system of claim 37, wherein one or more of said liquid handlers are configured to automatically remove lipid-containing solution and RNA-containing solution from a well plate.
